(19) United States
(12) Patent Application Publication
(10) Pub. No.: US 2010/0254510 A1
(43) Pub. Date: Oct. 7, 2010

(54) APPARATUS FOR DIAGNOSING BREAST CANCER BY NESTED FILTERS COATED MULTILAYER

(75) Inventors: Kwon-Ha Yoon, Jeollabuk-do (KR); Kwon-Soo Cheon, Gyeongsangnam-do (KR)

Correspondence Address: RABIN & Berdo, PC, 1101 14TH STREET, NW, SUITE 500, WASHINGTON, DC, 20005, US

(73) Assignee: Wonkwang University Center for Industry- Academy Cooperation, Jeollabuk-do (KR)

(21) Appl. No.: 12/280,294

(22) PCT Filed: Feb. 21, 2006
(86) PCT No.: PCT/KR2006/000570

§ 371 Date: Jun. 23, 2010

(51) Int. Cl. A61B 6/04; A61B 6/00

(52) U.S. Cl. 378/37

(57) ABSTRACT

Provided is a breast cancer diagnosing apparatus using an X-ray, in which the breast cancer diagnosing apparatus selects and uses energy within a specific band width through an energy band pass filter. In the breast cancer diagnosing apparatus, a multilayer filter unit having one or more layers of a basic filter having a multilayer structure is inserted between an X-ray generator and a breast fixing unit having a paddle fixing the breast so as to allow a predetermined band of an X-ray spectrum generated from the X-ray generator to pass therethrough and be emitted to the breast. According to the present invention, the X-ray dosage of the patient by energy within a low energy band (8-15 keV) can be reduced 5 times as compared with that by the conventional apparatus.

### TECHNICAL FIELD

The present invention relates to a breast cancer diagnosing apparatus. More particularly, the present invention relates to a breast cancer diagnosing apparatus that has a plurality of energy bandpass filters provided in the form of a multilayer film structure to improve an image quality and minimize X-ray dosages.

### BACKGROUND ART

Generally, a breast cancer diagnosing apparatus using mammography includes a rotating target type X-ray generator. Molybdenum or rhodium is mainly used as a target material. A maximum tube voltage applied to an anode of the X-ray generator is 40 kVp and electron beams emitted from a filament of the X-ray generator collide with the anode to generate X-rays by which the diagnosing image is taken.

In this case, the energy band except for a range of 15-20 keV where a contrast between a soft-tissue and a cancer in the X-ray image of the breast is excellent does not affect on an actual image. Especially, energy within a lower energy band (8-15 keV) is mostly absorbed in the breast to increase the X-ray dosage. Energy within a high energy band (20 keV or more) causes blurring of the image due to Compton scattering.

Meanwhile, in order to minimize the X-ray dosage of the breast by the energy within the low energy band, a filter such as an aluminum filter, a molybdenum filter, and a rhodium filter is used to select energy within a specific energy band. However, in this case, since a dose of a characteristic X-ray (e.g., 17.48 keV (Mo target)) that is effective for the image is reduced by the filter, the X-ray irradiation time must be increased and the image quality is not clear.

Recently, in order to select the characteristic X-rays, an image apparatus using monochromatic beam of X-rays at a Bragg angle through a silicon single crystal has been developed. In this image apparatus, a bandwidth of a specific energy is narrow and thus the dosage and scattering of the X-ray can be reduced. However, since a photon flux is too weak, it takes a long time to obtain the image.

### DISCLOSURE

### Technical Problem

The present invention has been made in an effort to solve the above-described problems of the related art. An object of the present invention is to provide a breast cancer diagnosing apparatus using a multilayer filter, which can (a) minimize reduction of characteristic X-rays, (b) minimize a dosage of the X-rays by filtering low and high energies using a single filter, and (c) improve an image obtained, thereby enabling early treatment of the breast cancer at an early stage and improving an image reading rate.

### Technical Solution

To achieve the object, the present invention provides a breast cancer diagnosing apparatus including an X-ray generator, a collimator for limiting a direction of the X-rays emitted from the X-ray generator at a predetermined angle to a vertical and/or horizontal direction, a multilayer filter unit for reflecting energy of a specific energy band of the X-rays, which are incident at the angle limited by the collimator, at a specific angle, a breast fixing unit including a paddle for fixing the breast to which beams reflected from the multilayer filter unit are emitted, an image capture unit having a width equal to or greater than a width of the beams reflected from the multilayer filter unit to capture a complete image of the breast by combining a pre-scanning image obtained and a post-scanning image, and a linear motion guide for fixing the X-ray generator, the collimator, the multilayer filter unit, and the image capture unit and moving them together or for moving only the breast fixing unit to perform the scanning work for obtaining an image of a vacuum portion caused by the stacked structure of the multilayer filter unit during the irradiation of the X-ray.

### ADVANTAGEOUS EFFECTS

In the breast cancer diagnosing apparatus using the multilayer filter according to the present invention, since the image that is identical to that obtained by a conventional apparatus can be realized by irradiating energy within a specific energy band (15-20 keV) to the breast, the X-ray dosage of the patient by energy within a low energy band (8-15 keV) can be reduced 5 times as compared with that by the conventional apparatus. In addition, since energy within 15-22 keV is allowed to pass, the contrast between a normal-tissue and a cancer-tissue can be improved two times as compared with that provided by the conventional apparatus. Furthermore, the image blurring phenomenon caused by the Compton scattering generated the energy within the high energy band (25-40 keV) can be prevented.

Particularly, when the high quality image where the contrast is improved is obtained, the disease identification can be accurately realized and the early diagnosis and early treatment becomes possible.

### DESCRIPTION OF DRAWINGS

FIG. 1 is a block diagram illustrating a breast cancer diagnosing apparatus using a multilayer filter according to an embodiment of the present invention;

FIG. 2 is a perspective view of a breast cancer diagnosing apparatus using a multilayer filter according to an embodiment of the present invention; and

FIG. 3 is a photograph taken by a breast cancer diagnosing apparatus using a multilayer filter according to the present invention.

[Description of symbols in main parts of the drawings]

| | |
|---|---|
| 1: X-RAY GENERATOR | 2: COLLIMATOR |
| 3: MULTILAYER FILTER UNIT | 4: BREAST FIXING UNIT |
| 5: IMAGE CAPTURE UNIT | 6: LINEAR MOTION GUIDE |

### BEST MODE FOR CARRYING OUT THE INVENTION

The following will describe an embodiment of the present invention in more detail with reference to the accompanying drawings.

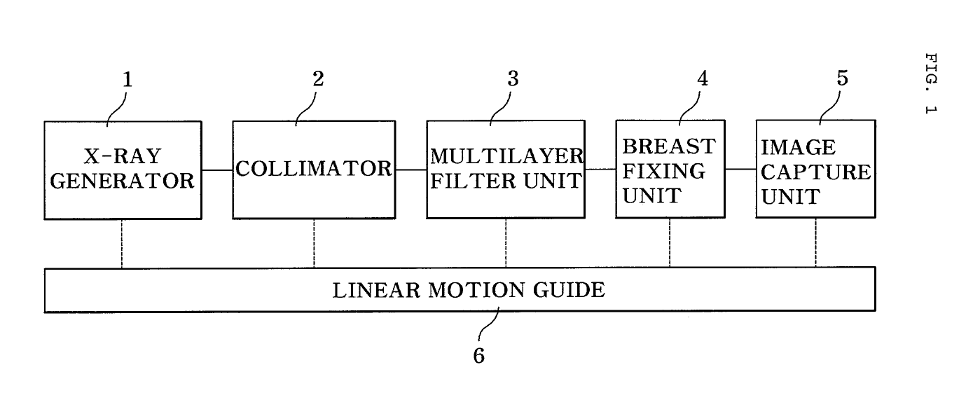

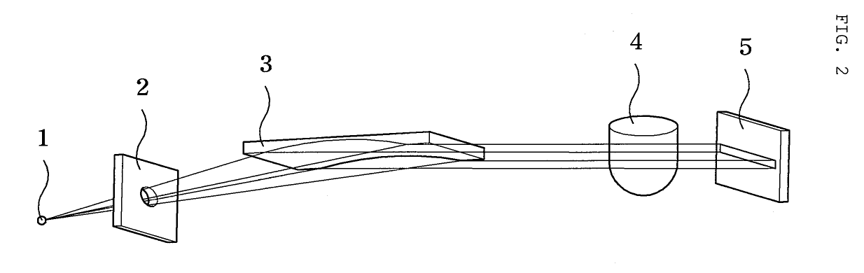

Referring to FIGS. 1 and 2, an X-ray generator 1 generates X-rays and irradiates the X-rays to the breast. Like the conventional X-ray generator, the X-ray generator 1 uses molybdenum or rhodium as a target material. In addition, when the X-ray generator 1 uses an identical excessive current/voltage condition to the conventional X-ray generator, it generates an emission spectrum that is same as that generated by the conventional X-ray generator.

A collimator 2 functions to limit a direction of the X-rays emitted from the X-ray generator 1 at a predetermined angle to a vertical and/or horizontal direction, thereby minimizing a dose of the X-rays that leaks to the patient or breast.

The collimator 2 allows the X-rays emitted from the X-ray generator 1 to be incident only on a multilayer filter 3. The collimator 2 is configured having a predetermined angle defined between extending lines of emission and incident angles to minimize scattering and formed of tungsten.

The multilayer filter unit 3 functions to reflect energy of a specific energy band of the X-rays, which are incident at the angle limited by the collimator 2, at a specific angle.

The multilayer filter unit 3 reflects the energy within the specific energy band (15-20 keV) among the X-rays, which are generated from the X-ray generator 1 and incident at the angle limited by the collimator 2, at the specific angle.

The multilayer filter unit 3 includes a plurality of basic filters each having a multilayer thin film structure. The basic filters are arranged in parallel/series to minimize a scanning time by enlarging an emission region to an area that can emit the X-rays to an entire region of the breasts and allowing the X-rays to overlap by a predetermined length.

The multilayer filter unit 3 is assembled such that, when the basic filters are aligned to be stacked one another, the basic filters except for the uppermost basic filter are disposed right under an extension line of a beam incident on a longitudinal end of the uppermost basic filter.

A breast fixing unit 4 includes a paddle for fixing the breast to which the beams reflected from the multilayer filter unit 3 are emitted.

An image capture unit 5 has a width equal to or greater than a width of the beams reflected from the multilayer filter unit 3 to capture a complete image of the breast by combining a pre-scanning image obtained and a post-scanning image.

A digital detector using a charge coupled device (CCD) or a complementary metal-oxide semiconductor (CMOS) camera having a width that is equal to or greater than that of the beams reflected from the multilayer filter unit 3 may be used as the image capture unit 5, thereby minimizing the price of the components and maximizing the utilization of the exposure dose.

A linear motion guide 6 fixes the X-ray generator 1, the collimator 2, the multilayer filter unit 3, and the image capture unit 5 and moves them together. Alternatively, the linear motion guide 6 moves only the breast fixing unit 4 to perform the scanning work for obtaining an image of a vacuum portion caused by the stacked structure of the multilayer filter unit 3 during the irradiation of the X-ray.

The following will describe an operation of the above-described breast cancer diagnosing apparatus using the multilayer filter.

First, when the X-ray generator 1 generates the X-rays in a state where all of the X-ray generator 1, the collimator 2, the multilayer filter unit 3, the breast fixing unit 4, and the image capture unit 5 are fixed by the linear motion guide 6, the collimator 2 limits a direction of the X-rays emitted from the X-ray generator 1 at a predetermined angle to a vertical and/or horizontal direction, thereby directing the X-rays to the multilayer filter unit.

When the X-rays whose directional angle is limed by the collimator 2 are incident on the multilayer filter unit 3, the multilayer filter unit 3 reflects energy of a specific energy band of the X-rays at a specific angle. Accordingly, the image capture unit 5 having a width that is equal to or greater than a width of the beams reflected from the multilayer filter unit 3 at a rear end of the breast fixing unit 4 captures the image of the breast.

At this point, the breast image captured by the image capture unit 5 is a pre-scanning image that is captured in a state where all of the X-ray generator 1, the collimator 2, the multilayer filter unit 3, the breast fixing unit 4, and the image capture unit 5 are fixed by the linear motion guide 6.

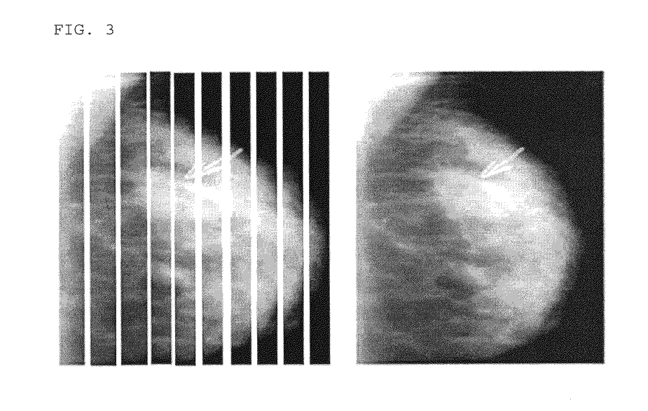

As shown by a left picture of FIG. 3, in the pre-scanning image, the vacuum portion caused by the stacked structure of the multilayer filter unit 3 during the irradiation of the X-ray is represented with a white color.

When the pre-scanning image is captured, an image of the vacuum portion represented with the white color is captured and scanned by the linear motion guide 6 to provide a complete breast image.

The scanning work may be performed by fixing and moving the X-ray generator 1, the collimator 2, the multilayer filter unit 3, and the image capture unit 5 together in a state where the breast fixing unit 4 is fixed so that the X-rays can be emitted to the breast fixed by the breast fixing unit 4. Alternatively, the scanning work may be performed by moving lonely the breast fixing unit 4 in a state where the X-ray generator 1, the collimator 2, the multilayer filter unit 3, and the image capture unit 5 are fixed not to move so that the X-rays can be emitted to the breast fixed by the breast fixing unit 4.

When the post-scanning image is captured by the image capture unit 5 through the above scanning work, a scanning image combination software installed in the image capture unit 5 is run so that the image capture unit 5 can combine the pre-scanning image and the post-scanning image so that the complete breast image can be obtained as shown in a right picture of FIG. 3.

Although embodiments have been described with reference to a number of illustrative embodiments thereof, it should be understood that numerous other modifications and embodiments can be devised by those skilled in the art that will fall within the spirit and scope of the principles of this disclosure.

1. A breast cancer diagnosing apparatus comprising:
an X-ray generator;
a collimator for limiting a direction of the X-rays emitted from the X-ray generator at a predetermined angle to a vertical and/or horizontal direction;
a multilayer filter unit for reflecting energy of a specific energy band of the X-rays, which are incident at the angle limited by the collimator, at a specific angle;
a breast fixing unit including a paddle for fixing the breast to which beams reflected from the multilayer filter unit are emitted;
an image capture unit having a width equal to or greater than a width of the beams reflected from the multilayer filter unit to capture a complete image of the breast by combining a pre-scanning image obtained and a post-scanning image; and
a linear motion guide for fixing the X-ray generator, the collimator, the multilayer filter unit, and the image capture unit and moving them together or for moving only the breast fixing unit to perform the scanning work for obtaining an image of a vacuum portion caused by the stacked structure of the multilayer filter unit during the irradiation of the X-ray.

2. The breast cancer diagnosing apparatus of claim 1, wherein the collimator is configured having a predetermined angle defined between extending lines of emission and incident angles and formed of tungsten.

3. The breast cancer diagnosing apparatus of claim 1, wherein the specific energy band reflected by the multilayer filter unit is 15-20 keV.

4. The breast cancer diagnosing apparatus of claim 1, wherein the multilayer filter unit includes a plurality of basic filters each having a multilayer thin film structure, the basic filters being arranged in parallel/series.

5. The breast cancer diagnosing apparatus of claim 1, wherein the multilayer filter unit is assembled such that, when basic filters are aligned to be stacked one another, the basic filters except for an uppermost basic filter are disposed right under an extension line of a beam incident on a longitudinal end of the uppermost basic filter.

6. The breast cancer diagnosing apparatus of claim 1, wherein the image capture unit is a digital detector using a CCD (Charge Coupled Device) or a CMOS (Complementary Metal-Oxide Semiconductor).
